(19) United States
(12) Patent Application Publication
(10) Pub. No.: US 2014/0187923 A1
(43) Pub. Date: Jul. 3, 2014

(54) COLLIMATOR FOR IMAGING SYSTEM AND METHOD FOR USING THE SAME

(71) Applicant: GENERAL ELECTRIC COMPANY, Schenectady, NY (US)

(72) Inventors: Floribertus Philippus Martinus Heukensfeldt Jansen, Ballston Lake, NY (US); Bruno Hans Haider, Ballston Lake, NY (US)

(73) Assignee: GENERAL ELECTRIC COMPANY, Schenectady, NY (US)

(21) Appl. No.: 13/730,384

(22) Filed: Dec. 28, 2012

(51) Int. Cl. A61B 6/00

(52) U.S. Cl. CPC A61B 6/44
USPC 600/425

(57) ABSTRACT

The embodiments disclosed herein relate generally to a probe that may be inserted into a patient. In certain embodiments, the probe includes a housing, a collimator assembly, and a detector assembly. The collimator and detector assemblies may rotate along a central axis within the housing. In addition, in certain embodiments, an imaging system including the probe and a method of using the probe are provided.

### BACKGROUND

The subject matter disclosed herein relates to a collimator design as well as to a probe for use with single photon emission computed tomography (SPECT).

Diagnostic imaging technologies allow images of the internal structures of a patient to be obtained providing information about the function and integrity of the patient's internal structures. Diagnostic imaging systems may operate based on the transmission and detection of radiation through or from the patient. For example, X-ray based imaging techniques (such as mammography, fluoroscopy, computed tomography (CT), and so forth) typically utilize an external source of X-ray radiation that transmits X-rays through a subject and a detector disposed opposite the X-ray source that detects the X-rays transmitted through the subject. Other radiation based imaging approaches, such as single photon emission computed tomography (SPECT) or positron emission tomography (PET) may utilize a radiopharmaceutical that is administered to a patient and which results in the emission of gamma rays from locations within the patient's body. The emitted gamma rays are then detected and the gamma ray emissions localized.

SPECT imaging relies on the use of collimators to limit the paths taken by emitted gamma rays from the patient to a detector such that the origin of the emitted radiation can be accurately determined In particular, collimation allows emitted gamma rays traveling only in certain directions to impact the detector resulting in the ability to resolve details of the patient's internal structures. The proximity of the collimators to a patient's region of interest affects the resolution of the images. Accordingly, collimators that are in close proximity to the patient's region of interest generate the most useful images. However, in certain situations it may be difficult to image certain internal structures due to their location within the patient's body. Furthermore, anatomical components, such as the pelvis and/or the bladder, may attenuate or interfere with the emitted radiation. For example, imaging of the prostate suffers from poor resolution and low count statistics because it is difficult to get close to the prostate and the pelvis causes significant attenuation of the emitted radiation.

### BRIEF DESCRIPTION

In accordance with one embodiment a probe is provided. The probe may be inserted into a patient and includes a detector assembly configured to detect radioactive signals and to generate electrical signals in response to the detected radioactive signals. The probe also comprises a collimator assembly positioned on or above the surface of the detector assembly and a housing configured to enclose the detector assembly and the collimator assembly. The detector assembly and collimator assembly are capable of rotation along a central axis within the housing.

In another embodiment, an imaging system is provided. The imaging system includes a detector assembly configured to detect radioactive signals and to generate electrical signals in response to the detected radioactive signals. The imaging system also comprises a collimator assembly positioned proximate to the surface of the detector assembly and a housing to enclose the detector assembly and the collimator assembly. The detector assembly is capable of rotation along a central axis within the housing. The imaging system also comprises a position encoder configured to determine the position of the detector assembly relative to the housing. The imaging system also comprises a data acquisition system in communication with the detector assembly and the position encoder as well as a controller controlling operation of one or both of the data acquisition system and the detector assembly.

In a further embodiment, a method for using an imaging probe is provided. The method generally involves administering a radioactive agent to a patient. The imaging probe is inserted into a passage within the patient's body. A detector assembly within the probe is rotated such that collection of radiation data resulting from the decay of the radioactive agent is viewed from different angles. A radioactive signal is collimated through a collimator assembly, at least a portion of which is positioned at a non-perpendicular angle relative to the surface of the detector assembly.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a block diagram illustrating an embodiment of a general imaging system that may incorporate a probe, in accordance with an aspect of the present disclosure;

FIG. 2 is an illustration of an embodiment of a probe, in accordance with an aspect of the present disclosure;

FIG. 3 is an illustration of an embodiment of a probe with a detector assembly and a collimator assembly that incorporates a shield, in accordance with an aspect of the present disclosure;

FIG. 4 is an illustration of an embodiment of a probe having a detector assembly with a shield including an ultrasound detector;

FIG. 5 is an illustration of an embodiment of a probe having a detector assembly with a shield including an ultrasound detector and a coupling gel;

FIG. 6 is an illustration of an embodiment of a probe having a detector assembly with a shield including a coupling gel;

FIG. 7 is a planar illustration of a plurality of collimator rows having different slant angles, in accordance with the present disclosure; and

FIG. 8 is a flow diagram illustrating a method of using a probe, in accordance with the present disclosure.

### DETAILED DESCRIPTION

The present disclosure relates to a probe with a novel collimator and detector assembly capable of rotating and translating within the probe. In certain embodiments an imaging system that uses the probe, combined with rotation and translation of the collimator assembly and a detector assembly about a central axis of the probe, are discussed along with a method of use. Accordingly, the imaging system is capable of reconstructing a volumetric image with limited angle tomographic information.

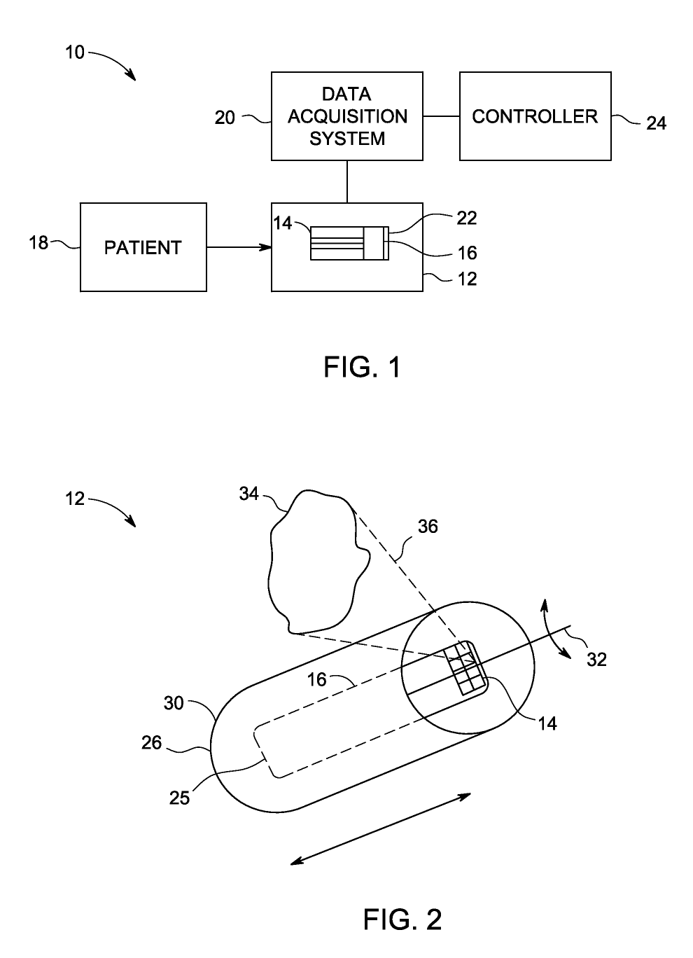

Referring now to FIG. 1, a block diagram illustration of a generalized imaging system 10 is provided. The imaging system 10 includes a probe 12 comprising a collimator assembly 14 and a detector assembly 16. The collimator assembly 14 acts to limit the direction from which a radioactive signal emitted within a patient 18 can strike the detector assembly 16 and be detected. It should be appreciated that the radioactive signal may be generated in response to the radioactive decay of a radioactive agent administered to a patient 18.

The collimator assembly 14 may be composed of an array of collimators positioned at multiple angles such that the detector assembly receives the radioactive signals from different view angles, as will be discussed in more detail below. The detector assembly 16 may include elements such as a detector, a position encoder, a motion actuator 22, and/or a radiation shield. In certain embodiments, the functionality of the position encoder and motion actuator may be combined. For example, the motion actuator 22 may be configured to provide position information (i.e., and/or translation information) for the detector assembly 16 without use of a separate and distinct position encoder.

The detector assembly 16 generates electrical signals in response to detected radiation, and these electrical signals are sent through their respective channels to a data acquisition system (DAS) 20. Once the DAS 20 acquires the electrical signals, which may be analog signals, the DAS 20 may digitize or otherwise condition the data for easier processing. For example, the DAS 20 may make corrections to the signal to correctly estimate the energy of a detected gamma photon, filter the image data for noise or other image aberrations, and so on. The DAS 20 then provides the data to a controller 24 to which it is operatively connected. The controller 24 may be an application-specific or general purpose computer with appropriately configured software. The controller 24 may include computer circuitry configured to execute algorithms such as imaging protocols, data processing, diagnostic evaluation, and so forth. As an example, the controller 24 may direct the DAS 20 to perform image acquisition at certain times, to filter certain types of data, and the like. Additionally, the controller 24 may include features for interfacing with an operator, such as an Ethernet connection, an Internet connection, a wireless transceiver, a keyboard, a mouse, a trackball, a display, and so on.

Turning now to FIG. 2, a schematic illustration of an embodiment of the probe 12 is provided. In the depicted example, the probe 12 comprises a housing 30 that encloses the collimator assembly 14 and the detector assembly 16 within the probe 12. The housing 30 may comprise medical grade (e.g., biocompatible) materials, such as, but not limited to, stainless steel, aluminum, silicone, or other suitable durable polymer or copolymer material, or a combination thereof that does not attenuate the radioactive signal emitted from the patient and which is suitable for use in an inserted or invasive device. In one embodiment the housing 30 has an outer or external diameter of approximately 2.8 cm, an internal diameter of about 2.0-2.5 cm and is approximately 8 cm long. Along a central axis 32 of the housing 30 is disposed the collimator assembly 14 and the detector assembly 16. In the depicted example, both the collimator and detector assemblies are capable of rotating and translating along the central axis 32 (such as due to operation of the motion actuator 22) such that the radioactive signal from a patient's region of interest 28 may be incident at different angles 36 and/or locations with respect to the detector assembly 16. In one embodiment, the detector assembly 16 extends along the central axis 32 such that a terminal end 25 of the detector assembly 16 may be positioned approximately 0.5-1.0 cm from a distal end 26 of the probe 12. In one such embodiment, the detector assembly 16 measures approximately 8 cm long, 2 cm wide and 4-5 mm thick. In one implementation, the detector assembly is a direct conversion detector that may include, but is not limited to, cadmium zinc telluride (CdZT), gallium arsenide, or mercuric iodide detection structures. It should be appreciated that the detector assembly may be configured as a single detector unit or multiple units (e.g., an array) positioned along the length of the detector assembly.

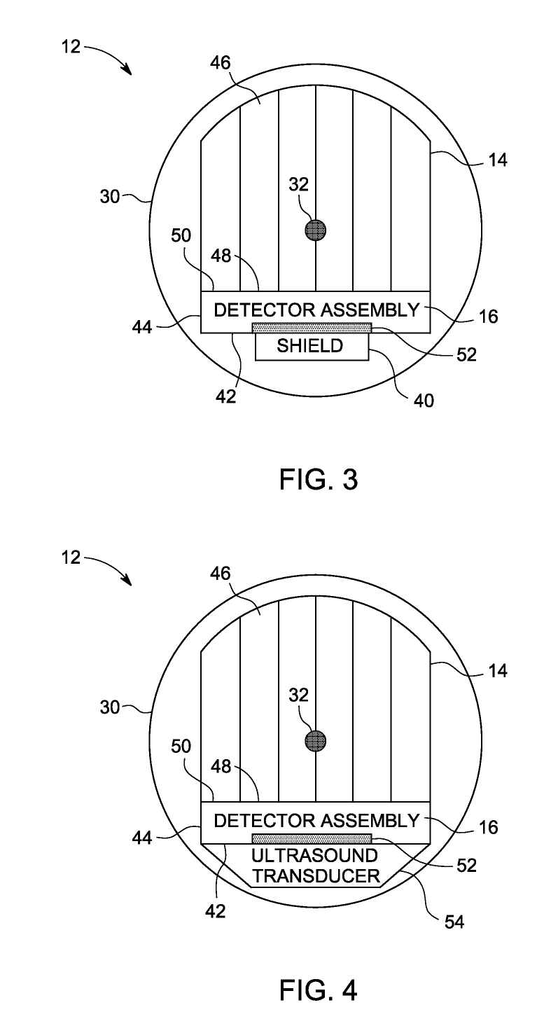

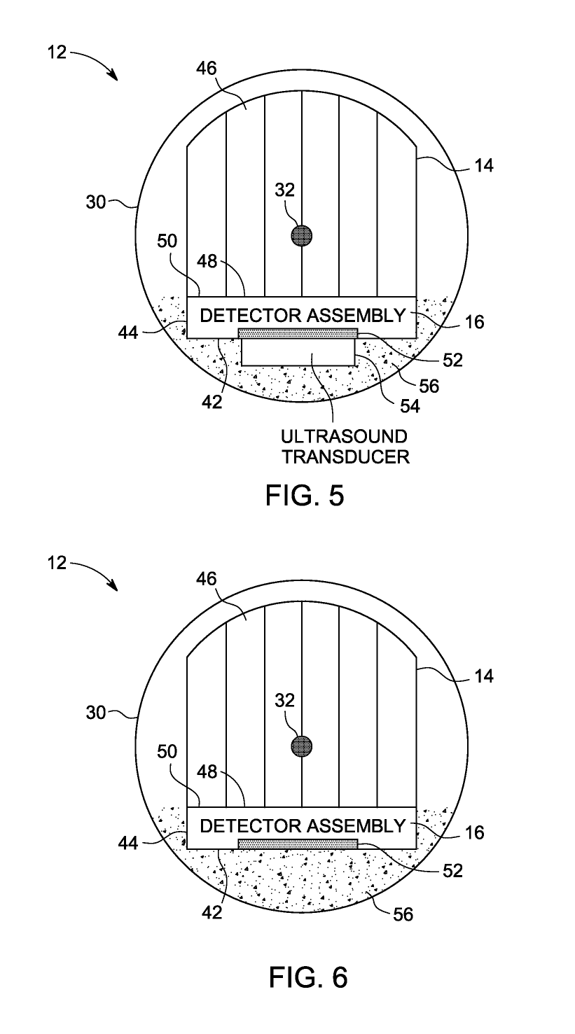

In the depicted example, the detector assembly 16 further includes a shield to minimize unwanted background and/or scattered radiation from reaching the radiation detector. Turning now to FIG. 3, a cross-sectional view of the probe 12 including a shield is depicted. In this example, the shield 40 is positioned on a bottom face 42 of the detector assembly 16. The shield function may, in some embodiments be performed by an ultrasound array (e.g., an ultrasound transducer array), an acoustic coupling gel, a highly radio-opaque material, such as tungsten, bismuth or lead, or a combination thereof. The coupling gel may include polyacrylamide based gels, but may also be loaded with nanoparticles of high Z materials such as tungsten, bismuth or lead, or chemical compounds including these and similar materials, in order to improve radiation attenuation at the back of the detector. In one embodiment illustrated in FIG. 4, an ultrasound array 54 may be provided which, in addition to providing ultrasound imaging functionality, also provides radiation shielding for all or part of the detector assembly 16 and may extend generally along the length and width of the detector assembly 16. By way of example, a suitable ultrasound array may include a piezoelectric transducer assembly or array. In another embodiment depicted in FIG. 5, the ultrasound array 54 may be provided in conjunction with a coupling gel 56 (e.g., an acoustic coupling gel) that itself provides some degree of radiation shielding and which may provide shielding at locations not shielded by the ultrasounds array 54, such as along the sides of the detector assembly 16. In a further embodiment shown in FIG. 6, the shielding function may be performed solely by the coupling gel 56 which may be provided in the space between the housing 30 and the detector assembly 16.

In certain embodiments, the ultrasound array 54, if present, may be used to image a patient's internal features to verify or facilitate proper placement of the probe within the patient. Accordingly, the detector assembly may be rotated such that the ultrasound array 54 is facing the patient's region of interest during such an ultrasound imaging process. Once the ultrasound imaging process is completed, the probe may be rotated and/or repositioned based on the acquired ultrasound image data so that the active surface of the detector assembly 16 faces the region of interest.

A position encoder 52 may also be present on the detector assembly 16 such that the position of the detector assembly 16 relative to the housing 30 (i.e., along axis 32) may be measured at any point and time. The position encoder may be provided as any suitable electromechanical measurement or control system (such as, but not limited to, optical or electromechanical measurement or control systems) that can be used to indicate the position of the detector assembly 16 with respect to axis 32 and/or housing 30.

In the depicted example, located along the surface of the detector assembly 16 is the collimator assembly 14. The collimator assembly 14 is formed from a plurality of radio-opaque septa that limit the directions or angles that a radioactive signal can travel and reach the detector assembly 16. For example, the collimator assembly 14 may be provided as rows and columns of collimator septa 46. The collimator septa 46 may be made from a radio-opaque material including, but not limited to, tungsten, lead, bismuth, tantalum, gold, or any other suitable materials including polymers and copolymers capable of attenuating gamma radiation to a sufficient degree. In certain implementations, the collimator septa have a thickness of between 0.2 and 0.5 mm such that a top surface 48 of the detector assembly 16 has minimal obstruction and penetration of gamma radiation through each collimator septa (e.g., <5%). The optimal design of the collimator depends, among other things, on the energy of the incident radiation. As such, design of the details of the collimator may take into account the radiopharmaceutical that will be used for a specific imaging task.

In one embodiment, the length of the collimator septa 46 is equal to or greater than the internal radius of the probe 12, though this may vary depending on the placement of the detector 16 relative to the axis 32, the thickness of the detector 16, the presence and/or thickness of any shielding or ultrasound devices, and so forth. In one such embodiment, the collimator septa have a length between 2.0-2.4 cm and are positioned generally parallel and orthogonal to the detector assembly 16, though certain of the septa 46 may be at a different angle relative to the surface of the detector assembly 16. For example, in one embodiment in accordance with the present disclosure, the collimator septa located along an outer edge 50 of the detector assembly 16 have a longer length and are oriented at an angle relative to the collimator septa located along the central axis 32 of the probe 12, which are perpendicular to the surface of the detector element 16. Accordingly, due to being angled, the collimator septa located on the outer edge 50 have a length similar to that of the collimator septa located along the central axis 32 despite having a shorter distance between the edge surface of the detector assembly 16 and the probe housing 30, as measured perpendicular to the surface of the detector assembly 16. Consequently, the collimator septa located on the outer edge 50 will have similar path length with respect to the transmission of the emitted gamma rays as the collimator septa located along the central axis 32. In another embodiment, the collimator septa on the outer edge 50 may have a length equal to or shorter than the collimator septa on the central axis 32.

The collimator septa 46 may consist of a plurality of generally radio-opaque channels that limit the directions from which emitted radiation can reach a particular portion of the detector assembly 16. For example, in one embodiment, each of the plurality of collimator septa is aligned with a corresponding pixel located on the surface of the detector assembly 16. The plurality of collimator septa may be formed as parallel channels, diverging or converging holes or channels, as pinholes, or as some combination thereof It should be appreciated that in the case of very small pixels the plurality of collimator septa may each align with multiple pixels on the surface of the detector assembly 16. The cross-section of the respective collimator septa channels may be round, hexagonal, square, triangular, or a combination thereof

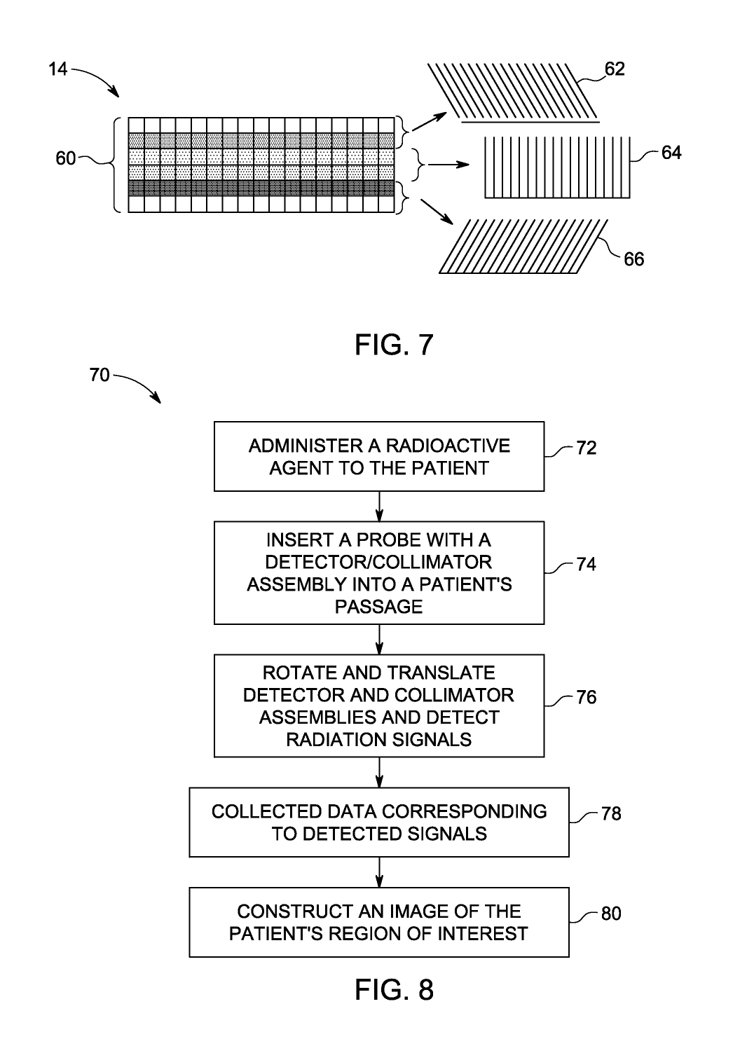

As noted above, the collimator septa are arranged in rows on the collimator assembly 14. In one implementation, different collimator rows are oriented at different angles relative to the detector assembly 16. To facilitate explanation, and turning to FIG. 7, a representative illustration of a collimator assembly 14 with six collimator rows 60, certain of which are slanted at different angles, is shown. For example, the collimator rows 60 may have a slant pattern of AABBCC, wherein A is a first angle 62, B is a second angle 64, and C is a third angle 66. In another embodiment, the collimator rows 60 may each have a different angle. For example, the angle pattern ABCDEF, wherein A is the first angle 62, B is the second angle 64, C is the third angle 66, D is a fourth angle (not shown), E is a fifth angle (not shown), and F is a sixth angle (not shown). In yet another embodiment in accordance with the present disclosure, the collimator rows may have repeating sequence of angles, ABCABC, or random order angles, CDFAEB, and any combination thereof It should be appreciated that the collimator assembly 14 can have any suitable number of collimator rows and is not limited to six and that the limited number of angles (and patterns) depicted is merely to simplify the present discussion. Furthermore, the angles can range from between −45° and +45° relative to the axis perpendicular to the front surface of detector assembly 16. As will be appreciated, as the range of angles gets larger, the detector may also need to be longer, and the maximum distance that may be imaged is shortened.

Positioning the collimator septa along rows 60 at an angle along certain positions of the detector assembly (such as the edges) allows data from the patient's region of interest to be collected from different view angles relative to the detector assembly 16 while maintaining a substantially constant path length through the collimator, such as in the depicted embodiments in which the collimator septa near the edge of the detector assembly 16 are angled relative to those near the center of the detector assembly 16. Further, rotation and translation of the collimator and detector assemblies allows different pixels of the radiation detector to view a particular voxel of the patient's region of interest from different angles, resulting in better depth resolution. The collimator and detector assemblies may be rotated slowly, wherein only a single pass is required, or may be rotated multiple times, such as to acquire data from different rotated positions for different displacements of the detector 16 along axis 32.

The position encoder is capable of measuring and simultaneously streaming the position of the detector assembly 16 relative to the axis 32, which can be transmitted along with the pixel readout data of the detector assembly. As such, continuous motion of the detector assembly 16 is permitted and allows for high resolution recovery during reconstruction of an image and zero dead time during acquisition of the data. Therefore, high sensitivity collimators having short collimator septa may be used, thus improving the sensitivity of the collimator assembly 14.

With the foregoing in mind, it should also be noted that, when a direct conversion detector (e.g., CZT) is used, the depth of interaction of the detected radiation may also be determined Depth of interaction in such a context may be established by measuring the ratio of anode to cathode signal or by measuring the rise time of the signals. For example, using a system as described herein, which has limited shielding (such as due to the finite size of the probe in order to allow for insertion into a body cavity), the expected distribution of depth of interaction for non-penetrating radiation may be known, allowing a comparison with the actual observed distribution to determine depth of interaction. By way of example, when radiation comes in through the front surface (after clearing the collimator), it might be expected that some percentage (e.g., 80%) of the radiation will be absorbed in the first 2.5 mm of a 5 mm detector (as will be appreciated, the percentage of absorption depends on the energy of the radiation and will thus vary based on this energy). Conversely, radiation coming in through the back surface would exhibit the opposite effect (for example, 80% of the radiation may be absorbed in the “bottom” 2.5 mm of a 5 mm detector).

With this in mind, statistical weighting of the observed events may be used to discriminate between events that entered the detector through the collimator from events that penetrated the shielding at the back of the detector. For example, assume equal numbers of counts are actually observed in the top and bottom half of the thickness of the detector. Based on the 80-20 expected distribution described in the example above, it may be concluded that there is as much radiation coming in through the back of the detector as through the front. But of the events in the top half of the detector, 80% may be deemed “good” and 20% may be deemed “bad”, while of events in the lower half 80% may be deemed “bad” and 20% deemed “good”, where “good” and “bad” refer to whether the detected event arises from a decay event associated with the breakdown of the radiopharmaceutical within the region of interest. Based on this knowledge, different weights may be allocated to the observed events based on their depth of interaction and the observed distribution. In this way, the depth of interaction information may be leveraged to provide additional information and certainty with respect to the results.

In accordance with the present disclosure, an embodiment for a method of use of the probe 12 is described. FIG. 8 is a block diagram illustrating a method 70 for using the probe 12. For example, the patient is first prepared to receive the probe 12. Accordingly, the patient is administered a radioactive agent that may include, but is not limited to, gallium-67, indium-111, iodine-123, technetium-99m, or a combination thereof (block 72). The probe 12 is then inserted into a suitable patient's passage (e.g. rectal cavity, vaginal cavity, trachea, esophagus, vasculature, and so forth) such that the patient's area of interest is in close proximity to the probe 12 (block 74). For example, if the patient's area of interest is the prostate, the probe 12 would be inserted into the patient's rectal cavity. It should be appreciated, that the housing 30 of the probe 12 may be inserted into a disposable sleeve that may include, but is not limited to, latex, rubber, and nitrile sleeves, prior to use with the patient. Moreover, lubricants that may facilitate insertion of the probe 12 into the patient's passage may also be used. Furthermore, the patient may be sedated or given local analgesics to provide comfort during an imaging procedure.

Once the probe 12 is inserted into the patient's passage, the collimator and detector assemblies are rotated and translated along the central axis of the probe 12 and the radiation signals from the patient's region of interest are detected (block 76). As described above, rotation and translation of the collimator and detector assemblies allows the radioactive signals from the patient's region of interest to be collected from a limited number of different view angles (block 78). A data set is generated from the radioactive signals, processed by the DAS and an image of the patient's area of interest is constructed (block 80).

This written description uses examples to disclose the subject matter, including the best mode, and also to enable any person skilled in the art to practice the disclosed approach, including making and using any devices or systems and performing any incorporated methods. It should also be understood that the various examples disclosed herein may have features that can be combined with those of other examples or embodiments disclosed herein. That is, the present examples are presented in such a way as to simplify explanation but may also be combined one with another. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A probe capable of being inserted into a patient comprising:
a detector assembly configured to detect radioactive signals and to generate electrical signals in response to the detected radioactive signals;
a collimator assembly positioned on or above the surface of the detector assembly; and
a housing configured to enclose the detector assembly and the collimator assembly, wherein the detector assembly and the collimator assembly are capable of rotation along a central axis within the housing.

2. The probe of claim 1, wherein the detector assembly is a radiation detector comprising a direct conversion material.

3. The probe of claim 2, wherein the direct conversion material comprises one or more of cadmium telluride (CdTe), cadmium zinc telluride (CZT or CdZnTe), gallium arsenide, or mercuric iodide.

4. The probe of claim 1, further comprising a position encoder capable of measuring a position of the detector assembly relative to the housing.

5. The probe of claim 1, wherein the collimator assembly comprises a plurality of collimator rows, wherein the plurality of collimator rows are each aligned with one or more pixels located on the surface of the detector assembly.

6. The probe of claim 1, further comprising a shield comprising a coupling gel, an ultrasound detector, or a combination thereof

7. The probe of claim 1, wherein the collimator assembly comprises a subset of collimator rows that are each slanted at a non-perpendicular angle relative to the detector assembly.

8. The probe of claim 7, wherein at least some of the collimator rows are slanted at angles different from other collimator rows.

9. The probe of claim 7, wherein the collimator rows each comprise respective collimator septa each configured to direct a radioactive signal at different view angles to the detector assembly.

10. The probe of claim 1, wherein the collimator assembly comprises a first subset of collimator septa located along a central axis of and positioned generally orthogonal to the detector assembly.

11. The probe of claim 1, wherein the collimator assembly comprises one or more subsets of collimator septa that are not orthogonal to the detector assembly.

12. An imaging system comprising:
a detector assembly configured to detect radioactive signals and to generate electrical signals in response to the detected radioactive signals; and
a collimator assembly positioned proximate to the surface of the detector assembly; and
a housing configured to enclose the detector assembly and the collimator assembly, wherein the detector assembly is capable of rotation along a central axis within the housing; and
a position encoder configured to determine the position of the detector assembly relative to the housing;
a data acquisition system in communication with the detector assembly and the position encoder; and
a controller controlling operation of one or both of the data acquisition system and the detector assembly.

13. The imaging system of claim 12, wherein the position encoder is provided as part of a motion actuator capable of moving at least the detector assembly with respect to the central axis.

14. The imaging system of claim 12, wherein the detector assembly comprises a radiation detector comprising a direct conversion material.

15. The imaging system of claim 14, wherein the direct conversion material comprises one or more of cadmium telluride (CdTe), cadmium zinc telluride (CZT or CdZnTe), gallium arsenide, or mercuric iodide.

16. The imaging system of claim 12, wherein the collimator assembly comprises a plurality of collimator rows comprising a plurality of collimator septa, wherein a first subset of the collimator septa are oriented orthogonal to the detector assembly and a second subset of the collimator septa are oriented at a non-orthogonal angle relative to the detector assembly.

17. The imaging system of claim 12, comprising a radiation shield comprising a coupling gel, an ultrasound detector, or a combination thereof

18. A method for using an imaging probe comprising:
administering a radioactive agent to a patient;
inserting the imaging probe into a passage within the patient's body;
rotating a detector assembly within the probe such that collection of radiation data resulting from the decay of the radioactive agent is viewed from different angles; and
collimating a radioactive signal through a collimator assembly.

19. The method of claim 18, wherein the probe is inserted into a patient such that the probe is proximal to an area of interest internal to the patient.

20. The method of claim 18, wherein the detector assembly is rotated inside the probe so that radiation signals enter a plurality of collimator septa of the collimator assembly such that the area of interest is viewed from a plurality of angles.

21. The method of claim 18, comprising constructing an image of the area of interest using one or more signals generated by the detector assembly.

22. The method of claim 18, comprising:
calculating a depth of interaction for the radioactive signal based on the expected distribution of depth of interaction for non-penetrating radiation;
determining a probability that an event is a non-penetrating event; and
weighting the event in an image reconstruction based on the probability.

23. The method of claim 22, wherein the detector assembly comprises a direct conversion detector.

24. The method of claim 18, wherein at least a portion of the collimator assembly is positioned at a non-orthogonal angle relative to the surface of the detector assembly.
